(19) United States
(12) Patent Application Publication
(10) Pub. No.: US 2023/0324393 A1
(43) Pub. Date: Oct. 12, 2023

(54) RAPID KIT FOR BREAST CANCER DIAGNOSIS BY USING MONOCLONAL ANTIBODY SPECIFIC TO TUMOR MARKER CANCER ANTIGEN 15-3

(71) Applicant: ADD BIOMEDICAL INC., Vancouver (CA)

(72) Inventor: Yonghyun PARK, Daejeon (KR)

(73) Assignee: ADD BIOMEDICAL INC., Vancouver (CA)

(21) Appl. No.: 17/715,614

(22) Filed: Apr. 7, 2022

(51) Int. Cl. G01N 33/574; G01N 33/543; B01L 3/00

(52) U.S. Cl. CPC G01N 33/57415; G01N 33/54388; B01L 3/5023; B01L 2300/069

(57) ABSTRACT

Disclosed is a diagnostic kit for diagnosis of breast cancer, the kit detecting the breast cancer tumor marker CA 15-3 in a biological sample, wherein the kit can diagnose the presence or absence of the breast cancer tumor marker CA 15-3 from test results conveniently and promptly without any special test equipment by using rapid immunochromatography using lateral flow assay, and thus has advantages of ease, economical efficiency, and speedy reading of test results.

### BACKGROUND OF THE INVENTION

### Field of the invention

This application relates to a rapid kit for breast cancer diagnosis by using a monoclonal antibody specific to tumor marker cancer antigen 15-3.

### Description of the Prior Art

A tumor marker is a biomarker that is secreted at a normal level or higher in blood, urine, or body fluids in the presence of a malignant tumor. The tumor marker is produced either by the tumor itself or by host cells in response to the tumor.

The American Society of Clinical Oncology (ASCO) recommends tests using tumor markers for the purpose of examination, screening, treatment, and prognosis observation of breast cancer. Typically, the expression analysis of cancer antigen 15-3 (CA 15-3), cancer antigen 27.29 (CA 27.29), carcinoembryonic antigen (CEA), estrogen receptor (ER), progesterone receptor (PR), human epidermal growth factor receptor 2 (HER2), urokinase plasminogen activator (uPA), plasminogen activator inhibitor 1 (PAI-1), or the like as a breast cancer marker shows clinical utility, and these are useful as breast tumor markers.

The breast cancer tumor marker CA 15-3, which is a transmembrane glycoprotein antigen, is originated from Mucine 1 (MUC1, cell surface-associated). CA 15-3 is also expressed in normal breast cells, but the production of CA 15-3 increases in many breast cancer patients. CA 15-3 does not cause cancer, but can be used as a useful tumor marker to track the cause of cancer since it is secreted from tumor cells and flows into the bloodstream. It is general that the progression of breast cancer tends to be more severe as the concentration of CA 15-3 in the blood increases, and the concentration thereof tends to increase as the size of the carcinoma increases. In addition, a very high concentration of CA 15-3 is observed in metastatic breast cancer resulting from the metastasis of cancer to the bone, liver, or the like, and is also observed in various diseases including liver cancer, pancreatic cancer, cirrhosis, and benign breast diseases. Even though the level of CA 15-3 is normal, CA 15-3 does not increase when an examination is performed very early, and normal CA 15-3 levels may be observed in 25-30% of metastatic breast cancer patients without the secretion of CA 15-3. Elevated CA 15-3 concentrations over time may mean that patients do not respond to treatment or cancer has recurred.

Therefore, the tumor marker CA 15-3 can be advantageously used as a breast cancer marker, and especially, can be used for the purposes of examination, screening, treatment, and prognosis observation of breast cancer. Korean Patent Application No. 2008-0100353 discloses a method for treating a neoplastic disease, including an antibody or peptide that specifically binds to a cellular component including cancer antigen 15-3 (CA 15-3).

Therefore, in the present disclosure, an attempt was made to develop a rapid kit for simple diagnosis, to which an immunoassay using lateral flow assay is applied and which can identify the presence or absence of the breast cancer marker CA 15-3 in the blood.

Throughout the specification, many papers and patent documents are referenced and their citations are provided. The disclosures of cited papers and patent documents are entirely incorporated by reference into the specification, and the level of the technical field within which the present disclosure falls, as well as details of the present disclosure are explained more clearly.

### PRIOR ART DOCUMENTS

### Patent Document

Korean Patent Application No. 2008-0100353, Polypeptide-nucleic acid conjugate for immunoprophylaxis or immunotherapy for neoplastic or infectious disorders, 17 Nov. 2008

### SUMMARY OF THE INVENTION

An aspect of the present disclosure is to provide a rapid kit for breast cancer diagnosis, the kit being useful for the diagnosis of breast cancer by more promptly identifying the presence of CA 15-3, a main tumor marker of breast cancer in a biological sample.

The present disclosure was made by establishing that the expression of the breast cancer tumor marker CA 15-3 in a biological sample can be promptly and accurately detected by using a simple diagnostic strip including: a gold conjugate pad including a conjugate of gold and a monoclonal mouse anti-CA 15-3 antibody binding to CA 15-3 antigen in a sample; and a membrane in which a test line containing an anti-CA 15-3 antibody immobilized therein and a control line for identifying the development of a sample are fixed.

Therefore, an aspect of the present disclosure is to provide a rapid kit for simple diagnosis of breast cancer by identifying the presence or absence of the breast cancer tumor marker CA 15-3.

Another aspect of the present disclosure is to provide a method for diagnosing breast cancer by using the kit of the present disclosure.

In accordance with an aspect of the present disclosure, there is provided a rapid kit for breast cancer diagnosis, the rapid kit including:
(a) a sample pad configured to allow a biological sample to be absorbed therein;
(b) a gold conjugate pad having one end overlapping one end of the sample pad, the gold conjugate pad containing a conjugate of gold and a monoclonal CA 15-3 antibody specifically binding to tumor marker CA 15-3 in a sample;
(c) a membrane having one end overlapping the other end of the gold conjugate pad, the membrane sequentially including: a test line in which a monoclonal CA 15-3 antibody specifically binding to tumor marker CA 15-3 is immobilized; and a control line in which a control antibody is immobilized; and
(d) an absorbent pad having one end overlapping the other end of the membrane to be capable of absorbing a liquid sample from the membrane.

The immunochromatography used in the rapid kit for breast cancer diagnosis, which is generally a diagnostic method enabling the examination of various diseases by using an antibody-antigen reaction, is a type of immunoassay for prompt on-site diagnosis wherein a porous thin film serving capillary action is in a solid phase and colloidal gold or colored particles such as colored polystyrene nanoparticles is used as a measurement label.

In the rapid kit for breast cancer diagnosis of the present disclosure, the “sample pad” configured to allow a biological sample to be absorbed refers to a pad that receives a sample to be analyzed to enable the diffusive flow of the sample, and is composed of a material having sufficient porosity to receive and contain the sample to be analyzed. Examples of the porous material include fibrous paper, a microporous membrane made of a cellulosic material, cellulose, a cellulose derivative such as cellulose acetate, nitrocellulose, a glass fiber, naturally occurring cotton, fabrics such as nylon, or a porous gel, but are not limited thereto.

In the rapid kit for breast cancer diagnosis, the “gold conjugate pad” receives a sample that is diffused to migrate from the sample pad as well as contains a conjugate of gold and a monoclonal CA 15-3 antibody specifically binding to the tumor marker CA 15-3. The gold conjugate pad, like the sample pad, is composed of a material enabling diffusive flow.

In the kit of the present disclosure, the “membrane” may be prepared of any variety of materials through which a sample material can pass. For example, the membrane may be formed of natural, synthetic, or naturally occurring materials that are synthetically modified, such as polysaccharides (e.g., cellulose materials, paper, and cellulose derivatives, such as cellulose acetate and nitrocellulose); polyether sulfone; polyethylene; nylon; polyvinylidene fluoride (PVDF); polyester; polypropylene; silica; inorganic materials uniformly dispersed in a porous polymer matrix, with polymers such as vinyl chloride, vinyl chloride-propylene copolymer, and vinyl chloride-vinyl acetate copolymer, for example, deactivated alumina, diatomaceous earth, MgSO4, or other inorganic finely divided material; cloth, naturally occurring (e.g., cotton) and synthetic (e.g., nylon or rayon); porous gels, such as silica gel, agarose, dextran, and gelatin; polymeric films, such as polyacrylamide; and so forth.

Preferably, the membrane contains polymeric materials, such as nitrocellulose, polyether sulfone, polyethylene, nylon, polyvinylidene fluoride, polyester, and polypropylene.

In the kit of the present disclosure, the “absorbent pad” may be positioned at a distal end of the membrane or adjacent to the distal end of the membrane. The absorbent pad usually receives a fluid sample migrating through the entire membrane. The absorbent pad can help facilitate capillary action and diffusive flow of a fluid through the membrane.

The kit of the present disclosure is configured by sequentially connecting the sample pad, gold conjugate pad, membrane, and absorbent pad on the same support.

The monoclonal CA 15-3 antibody used in the rapid kit for breast cancer diagnosis of the present disclosure specifically binds to cancer antigen 15-3 with high affinity. As used herein, the term “monoclonal antibody” refers to an antibody molecule having a single molecular composition obtained from the substantially the same antibody population, and the monoclonal antibody shows single binding specificity and affinity for a specific epitope.

In the rapid kit for breast cancer diagnosis of the present disclosure, a biological sample, when applied to the sample pad, is developed to the sample pad, gold conjugate pad, test line and control line of the membrane, and the absorbent pad according to the capillary action. Therefore, the sample pad, the gold conjugate pad, the membrane, and the absorbent pad are disposed in that order in the rapid kit for breast cancer diagnosis.

An anti-mouse IgG was used as the control antibody used in the rapid kit for breast cancer diagnosis of the present disclosure, and a different control antibody may be used depending on the production characteristics of the monoclonal CA 15-3 antibody.

The biological sample used in the rapid kit for breast cancer diagnosis of the present disclosure is human whole blood or plasma or serum isolated from whole blood.

In accordance with another aspect of the present disclosure, there is provided a method for providing information for breast cancer diagnosis by using the rapid kit for breast cancer diagnosis, the method including:
(a) allowing a biological sample to be absorbed into the sample pad of the rapid kit for simple diagnosis of breast cancer;
(b) allowing the biological sample to be developed to the gold conjugate pad, the membrane, and the absorbent pad from the sample pad absorbing the biological sample; and
(c) identifying the signals from the test line and the control line of the membrane to determine the presence or absence of tumor marker CA 15-3.

In step (c), the tumor marker CA 15-3 is determined to be present if color bands are shown in the test line and the control line of the membrane, and the tumor marker CA 15-3 is determined to be absent if a color band is shown in only the control line.

In accordance with another aspect of the present disclosure, there is provided a diagnostic method using the kit for simple diagnosis of breast cancer, the kit detecting the breast cancer tumor marker CA 15-3.

The present disclosure is directed to a kit for simple diagnosis of breast cancer, the kit detecting the breast cancer tumor marker CA 15-3 in a biological sample. The kit for simple diagnosis of breast cancer of the present disclosure is performed by immunochromatography using lateral flow assay. Specifically, the kit of the present disclosure is an application of the immunological reactivity of an antibody on the breast cancer tumor marker CA 15-3 as an antigen, the color development properties of gold particles (colloidal gold), fluidity, and the migration of molecules by membrane capillary action. This rapid immunochromatography can be used to diagnose the presence or absence of the breast cancer tumor marker CA 15-3 from the test results conveniently and promptly without any special test equipment, and thus has advantages of ease, economical efficiency, and speedy reading of test results.

### BRIEF DESCRIPTION OF THE DRAWINGS

FIG. 1 is a schematic diagram showing a strip for rapid immunochromatography of the present disclosure.

FIG. 2 depicts sensitivity according to the concentration of the tumor marker CA 15-3 in a strip for rapid immunochromatography of the present disclosure. 1. CA 15-3 50 U/ml, 2. CA 15-3 25 U/ml, 3. CA 15-3 12.5 U/ml, 4. CA 15-3 6.25 U/ml, 5. CA 15-3 0U/ml.

FIG. 3 depicts the detection limit for the breast cancer tumor marker CA 15-3 antigen in a strip for rapid immunochromatography of the present disclosure. 1. CA 15-3 40 U/ml, 2. CA 15-3 35 U/ml, 3. CA 15-3 30 U/ml, 4. CA 15-3 25 U/ml, 5. CA 15-3 20 U/ml, 6. CA 15-3 15 U/ml, 7. CA 15-3 10 U/ml.

### DETAILED DESCRIPTION OF THE EXEMPLARY EMBODIMENTS

The present disclosure is directed to a diagnostic kit including a test strip for detecting the breast cancer tumor marker CA 15-3, the test strip including: (a) a sample pad configured to allow a sample to be absorbed; (b) a gold conjugate pad containing a conjugate of gold and a monoclonal mouse anti-CA 15-3 antibody binding to the breast cancer tumor marker CA 15-3 antigen in a sample; (c) a test membrane including a test line in which an anti-CA 15-3 antibody is immobilized and a control line in which anti-mouse IgG is immobilized; and (d) an absorbent pad configured to allow the residual sample to be absorbed.

In the diagnostic kit, the sample pad, the gold conjugate pad, the test line, the control line, and the absorbent pad are preferably disposed in that order, wherein 0.1-10 μg of the monoclonal mouse anti-CA 15-3 antibody is preferably immobilized in the test line, but is not limited thereto. The conjugate of gold and a monoclonal mouse anti-CA 15-3 antibody is preferably dispensed at 0.01-10 μg in the gold conjugate pad, but is not limited thereto. The control line is preferably treated with 0.1-10 μg of anti-mouse IgG, but is not limited thereto. A sample used in the diagnostic kit is whole blood, serum, or plasma, but is not limited thereto.

The diagnostic kit shows a sensitivity of at least 95% and a specificity of at least 95% in the detection of the breast cancer tumor marker CA 15-3, wherein the concentration of the CA 15-3 antigen detected is 30 U/ml or more.

In addition, the present disclosure is directed to a method for diagnosing breast cancer by using the diagnostic kit to identify the expression of the breast cancer tumor marker CA 15-3. The CA 15-3 diagnostic method of the present disclosure is characterized in that specifically, when a sample is placed into the sample pad of the kit for breast cancer diagnosis, the sample is determined to be suspected of breast cancer if color bands are shown on the test line and control line of the test strip, and the sample is determined to be negative in the suspicion of breast cancer if a color band is shown on only the control line. In the kit for breast cancer diagnosis of the present disclosure, the purely purified monoclonal anti-CA 15-3 antibody is dispensed at an appropriate concentration in the test line region of the membrane, and monoclonal anti-mouse IgG specifically binding to mouse IgG is dispensed in the control line region of the membrane. Therefore, when an appropriate amount of an unknown sample is allowed to react with the kit, the sample performs a primary antigen-antibody reaction with the monoclonal anti-CA 15-3-gold conjugate of the conjugate pad depending on the presence or absence of the breast cancer tumor marker CA 15-3 antigen in the sample, flows by capillary action of the membrane, performs a secondary antigen-antibody reaction with the monoclonal antibody dispensed in the test line region, and finally, binds with mono-clonal anti-mouse IgG in the control line region, and thus the test is terminated. After the termination of the reaction, the presence or absence of the breast cancer tumor marker CA 15-3 in the sample is read out depending on the presence or absence of a color band on the test line.

In the present disclosure, the sensitivity is the sum of the probabilities that a test result is positive in the test among samples that are actually positive, and can be used as an indicator to identify to what extent the test can sort a positive sample.

The accuracy refers to the ratio of, relative to all samples, the sum of the number of cases where an actual positive sample is determined to be positive and the number of cases where an actual negative sample is determined to be negative.

Hereinafter, the present disclosure will be described in more detail with reference to examples. These examples are only for illustrating the present disclosure more specifically, and it will be apparent to those skilled in the art that the scope of the present disclosure is not limited by these examples.

### EXAMPLE 1: PREPARATION OF ANTIGEN AND ANTIBODY FOR DEVELOPMENT OF ANTI-CA 15-3 DIAGNOSTIC KIT

### (1) CA 15-3 Antigen

As a standard material for selecting antibodies and fabricating a diagnostic kit to diagnose the presence or absence of the breast cancer tumor marker CA 15-3 in a human blood sample, CA 15-3 (Order code 143404) isolated from patient sera was purchased from Laboratory Corporation of America Holdings.

Quantification: The concentration of the antigen was 30.0 U/ml, measured by Roche Diagnostics Electrochemiluminescence Immunoassay (ECLIA).

### (2) Antibody

To prepare a conjugate of gold and an antibody to be immobilized on the membrane for the diagnosis of the breast cancer positive marker CA 15-3, the anti-cancer antigen 15-3 (CA 15-3) mouse monoclonal antibody (mAb) was purchased from Genemedi (Cat. No. GMP-CA15-3-Ab-1).

Quantification: The lyophilized antibody was dissolved in tertiary distilled water to be made into 1 mg/ml, which was an appropriate concentration standard for line dispensing. Thereafter, the absorbance was measured at A280 by using a spectrometer, and the concentration was confirmed to be the same as the indicated antibody concentration.

### EXAMPLE 2: IDENTIFICATION OF ANTIGEN AND ANTIBODY REACTIVITY

To investigate the reactivity between the antigen and the antibody, a gold conjugate was formed to investigate the reactivity with the antigen immobilized on the membrane.

### (1) Antigen Immobilization

After CA 15-3 was dropped at 50 U, 25 U, 12.5 U, 6.25 U, and 0 U on the strips with the nitrocellulose membrane, the membranes were dried in a drying room in which 25-30° C. and a humidity condition of 20% or less were maintained.

(2) Preparation of antibody-gold particle conjugate

A gold conjugate was prepared in the condition of pH 9 for the antibody.

1) After 10 ml of 40-50 nm colloidal gold was titrated to pH 9.0 by using K₂CO₃, 1 ml of colloidal gold was taken and placed in a 1.5-ml tube.

2) Anti-CA 15-3 was diluted with a phosphate buffer to a concentration of 0.1 mg/ml, and then 0.1 ml was prepared.

3) The diluted antibody was placed in the tube in which the colloidal gold was placed, and then immediately vigorous mixing was performed using a vortex mixer for about 30 seconds, followed by reaction with gentle shaking for about 10 minutes.

4) After step 3), 100 μl of 5% casein was placed in the tube, and then immediately vigorous mixing was performed using a stirrer for about 30 seconds, followed by reaction with gentle shaking for about 10 minutes.

5) After step 4), centrifugation was performed at 8,000 rpm for about 10 minutes at 4° C. by using a centrifuge, followed by supernatant removal, and then pellets were completely dissolved by addition of 1 ml of a 1% BSA/2 mM borax solution.

6) After step 5), centrifugation was performed at 8,000 rpm for about 10 minutes at 4° C. by using a centrifuge, followed by supernatant removal, and then pellets were completely dissolved by addition of a 1% BSA/borax solution to a final volume of 0.1 ml, thereby preparing an antibody-gold particle conjugate.

### (3) Identification of Antigen-Antibody Reactivity

The strips having the antigen immobilized therein at 50 U, 25 U, 12.5 U, 6.25 U, and 0 U and the antibody-gold conjugate were used to analyze the degree of reactivity between the antigen and the antibody. To this end, 2 μl of the prepared antibody-gold conjugate and 48 μl of a buffer (50 mM borate pH 9.3, 0.5% casein, 50 mM NaCl, 2% Tween 20) were placed in a 1.5-ml tube, followed by mixing, and then the strips with the antibody adsorbed thereon were inserted to perform reaction for 5 minutes, and thereafter the results were checked. The prepared antibody-gold conjugate reacted with the antigen immobilized on the strips, and the color development was shown in the samples in which the antigen was dropped at 50 U and 25 U.

### EXAMPLE 3: FABRICATION OF RAPID KIT FOR BREAST CANCER DIAGNOSIS BY USING RAPID IMMUNOCHROMATOGRAPHY

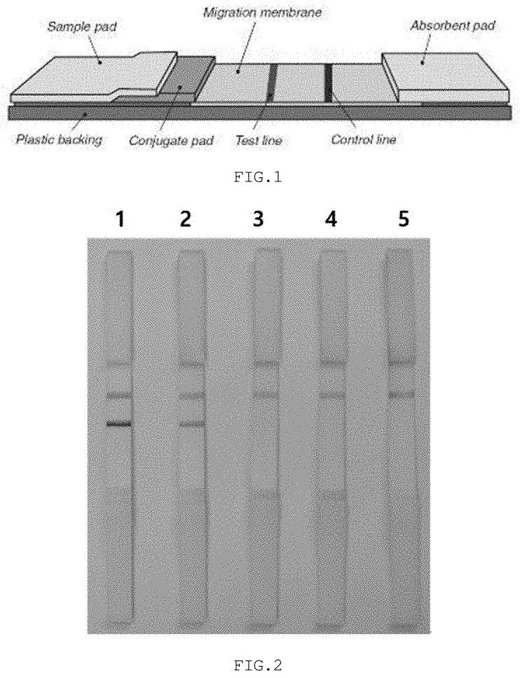

As shown in FIG. 1, a strip was fabricated by overlapping a sample pad, a gold conjugate pad, a membrane, and an absorbent pad to have a form in which a reaction with a capture occurred with flowing in a constant direction.

### (1) Immobilization of Antibody to Membrane

An antibody to be dispensed on the membrane was diluted with a phosphate buffer to different concentrations, thereby setting an optimal concentration for membrane immobilization for the antibody.

1) Setting dispensing conditions according to concentration

A nitrocellulose membrane was laminated onto a plastic card. Thereafter, monoclonal anti-CA 15-3 was dispensed in the test line region and monoclonal anti-mouse IgG was dispensed in the control line region, thereby setting dispensing conditions for enabling the adsorption to the membrane and making effective detection sensitivity.

As for antibodies for dispensing, an antibody for the test line and an antibody for the control line were made to have concentrations of 2 mg/ml and 1 mg/ml, respectively, and 0.5% sucrose and 0.1% NaN₃ were added thereto before dispensation.

2) Test on sensitivity to antigen

The membrane after dispensation was attached to the plastic card, and then a gold pad and a sample pad were attached thereto. The sensitivity was tested for each dispensation concentration of the concentrations (50, 25, 12.5, 6.25, and 0 U/ml) of CA 15-3. As shown in FIG. 2, antigen CA 15-3 was detected at the concentrations of 50 U/ml and 25 U/ml, but the color development was not shown at concentrations below that concentrations.

Membrane attachment and dispensation:

The membrane was attached to the plastic card, followed by dispensing in the following dispensing conditions, and then drying was performed for 48 hours in a drying room in which a humidity of 25% or lower was maintained.

(Dispensing conditions)

Control line: Monoclonal anti-mouse IgG, 1 mg/ml 1 μl

Test line: Monoclonal anti-CA 15-3, 2 mg/ml 1 μl

### (2) Antibody-Gold Conjugation

### 1) Setting Preparation Conditions Of Colloidal Gold

A predetermined amount of colloidal gold was dissolved in distilled water, and then sodium citrate as a reducing agent was added with vigorous stirring at 100° C., thereby manufacturing colloidal gold particles with a size of about 40 nm.

As a result of measuring the 0.D value of the manufactured colloidal gold by using a spectrometer, the highest value was shown at 540 nm, with O.D 540 =2.0.

2) Setting manufacturing conditions for gold particle conjugate

The colloidal gold solution was titrated to the conditions of pH 7.0, 8.0, and 9.0, and then monoclonal mouse anti-canine IL-6 (clone-247017) was conjugated to gold, and for finding the pH condition without a non-specific reaction, comparison tests were performed on ELISA-negative and -positive low titer samples. As a result, the weak but positive color development was shown in the weak-positive samples without differences among pH 7.0, 8.0, and 9.0 conditions, but the weak-positive results were shown for two negative samples in pH 7.0 conditions.

Although the results at pH 8.0 and pH 9.0 were almost similar, the antibody is known to have optimum conditions at pH 9.0, and thus stably, pH 9.0 was set as the optimum condition for manufacturing the antibody-gold conjugate.

### (3) Preparation of Conjugate Pad

The preparation of a conjugate pad includes: a conjugate pad pretreatment step; a gold conjugate solution dispensing and drying step; and a conjugate pad cutting step.

### 1) Conjugate Pad Pretreatment Step

The pretreatment of the conjugate pad was performed by sufficient wetting in a Tris buffer (20 mM, pH 8.0) containing 0.5% polyvinyl alcohol (PVA), which is an optimal condition set by varying the concentration of polyvinyl alcohol (PVA) and the concentration of the Tris buffer, and complete drying in a drying room.

The gold conjugation solution in which an appropriate concentration of an antibody is bound to colloidal gold particles having a diameter of about 40 nm was dispensed in the pre-treated conjugate pad, which was then completely dried in a drying room maintained at a humidity of 30% or less, and then cut according to the size (7 mm×300 mm).

### 2) Setting Preparation Conditions for Conjugate Pad

The gold conjugation solution was prepared by mixing the monoclonal anti-CA 15-3 gold conjugate with 1×PBS, 5% sucrose, and 0.5% casein to reach 0.D 540=6.

For the treatment of one sheet of 200 mm×300 mm polyester pad, 15 ml of the prepared gold conjugation solution was used. The treated pad was sufficiently dried for about 24 hours so that the humidity was maintained at 30% or less in the drying room.

### (4) Sample Pad Composition and Preparation

As for the sample pad, for the smooth development of a sample and the minimization of non-specific reactions, the sample pad was sufficiently wetted with 50 mM borate pH 9.3, 0.5% casein, 50 mM NaCl, 2% Tween 20 solution, and then completely dried in a dryer, and then cut according to the size (18 mm×300 mm).

### (5) Absorbent Pad Preparation

The absorbent pad was used itself without any treatment in a state in which moisture was completely dried, and for smooth absorption of a sample, the absorbent pad was cut to a size (18 mm×300 mm) that could overlap the membrane from the end of the plastic card.

### (6) Plastic Housing (Device)

A device was ejected in which no overflow occurred when a solution was added to the sample pad, especially, no overflow occurs even when 100-120 μl of a solution was placed in the sample pad; the maximum antigen-antibody reaction was induced when a sample was placed; the solution developed to the control line at a development rate of 130-180 seconds after the injection of a sample; and the test strip was mounted without shaking when the test strip was assembled.

### (7) Device Assembling

The conjugate pad, sample pad, absorbent pad, and test strip, which were prepared in the respective processes. That is, a test paper was completed by attaching the conjugate pad onto the lower end of the membrane to partially overlap the sample pad and attaching the absorbent pad to the upper end of the membrane, and the completed test paper was cut to about 4 mm by a cutter, thereby preparing a strip. Thereafter, the strip was placed and assembled in a plastic housing, and placed and packaged in an airtight container embedding an anti-moisture agent.

### (8) Test Method

The diagnostic kit of the present disclosure is a kit capable of identifying the presence or absence of the breast cancer tumor marker CA 15-3 at a certain concentration or more, and whole blood, serum, and plasma may be used as samples. The whole blood needs to be immediately used before clotting occurs, and the serum and plasma are refrigerated, and then used after being left at room temperature for about 30 minutes upon testing.

1) After the aluminum wrapper is opened, the test cassette is taken out, and then 100 pl of the sample is dropped onto the sample drop site by using an appropriate tool, such as a pipette.

2) The color development of the control line and test line was checked between 5 and 10 minutes.

3) The presence or absence of the breast cancer tumor marker CA 15-3 was diagnosed. However, the results appearing after 15 minutes is not included in the determination.

### (9) Result Determination and Analysis

The presence or absence of color bands in the test line (T) and control line (C) is checked to make a determination for positive or negative results. The control line (C) always appears regardless of the presence or absence of CA 15-3 in the sample, and this is for checking the presence or absence of abnormality in the reaction.

The color development is shown in the test line (T) in the presence of a predetermined concentration (30U/ml) or more of CA 15-3 in the sample, and the sample may be determined to be negative if CA 15-3 was present at a concentration below the predetermined concentration.

For example, the sample was determined to be negative when a color band was absent in the test line T and present only in the control line C, and the sample was determined to be positive when color bands were shown in the control line C and the test line T. If no color band was shown in the control line (C), the test was wrong or the quality of a reagent is problematic even through a color band appears in the test line (T), and therefore, a new reagent needs to be used to perform re-test.

### EXAMPLE 4: DIAGNOSTIC KIT EFFICACY TEST

### (1) Detection Limit on Breast Cancer Tumor Marker CA 15-3 Antigen

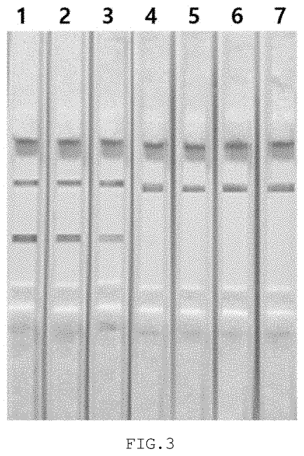

The results of repeated tests on the breast cancer tumor marker CA 15-3 in a phosphate buffer at 40 U/ml, 35 U/ml, 30 U/ml, 25 U/ml, 20 U/ml, 15 U/ml, and 10 U/ml are shown in Table 1 and FIG. 3. As shown in FIG. 3, the positive results were shown at all of the concentrations of 30 U/ml or more. As shown in Table 1, in the case of 25 U/ml, among three samples, one sample was determined to be positive and two samples were determined to be negative, and all the samples at concentrations below those concentrations showed negative results.

Therefore, the rapid kit for simple diagnosis of breast cancer by using the breast cancer tumor marker CA 15-3, which was a product in the present research results, had a detection limit of 30 U/ml.

TABLE 1

| Antigen concentration | First | Second | Third |
|---|---|---|---|
| 40 U/ml | Positive | Positive | Positive |
| 35 U/ml | Positive | Positive | Positive |
| 30 U/ml | Positive | Positive | Positive |
| 25 U/ml | Negative | Positive | Negative |
| 20 U/ml | Negative | Negative | Negative |
| 15 U/ml | Negative | Negative | Negative |
| 10 U/ml | Negative | Negative | Negative |

### (2) Sensitivity and Specificity

The sensitivity and specificity were measured by comparison of the present kit and the ELISA method, and are shown in Table 2.

TABLE 2

| | | | | | Measurement | |
|---|---|---|---|---|---|---|
| | | | | | sensitivity | |
| AbCam Human | | | | | (Rapid | |
| Cancer Antigen | | | | | positive/ | |
| CA15-3 ELISA | | Rapid Kit | | | ELISA | |
| Kit | | Positive | Negative | Sum | positive) | 96% |
| ELISA | Positive | 48 | 1 | 49 | Measurement | 98% |
| | Negative | 2 | 49 | 51 | specificity | |
| | | | | | (Rapid | |
| | | | | | positive/ | |
| | | | | | ELISA | |
| | | | | | positive) | |

As shown in FIG. 2, the rapid kit for breast cancer diagnosis according to the present disclosure, compared with the ELISA method, showed a high measurement sensitivity of 96% and a high measurement specificity of 98%, and thus it was identified that the present kit had effective cancer diagnosis performance.

Although the present disclosure has been described in detail with reference to the specific features, it will be apparent to those skilled in the art that this description is only for a preferred embodiment and does not limit the scope of the present disclosure. Thus, the substantive scope of the present disclosure will be defined by the appended claims and equivalents thereof.

What is claimed is:

1. A rapid kit for breast cancer diagnosis, the rapid kit comprising:
a) a sample pad configured to allow a biological sample to be absorbed therein;
b) a gold conjugate pad having one end overlapping one end of the sample pad, the gold conjugate pad containing a conjugate of gold and a monoclonal CA 15-3 antibody specifically binding to tumor marker CA 15-3 in a sample;
c) a membrane having one end overlapping the other end of the gold conjugate pad, the membrane sequentially comprising: a test line in which a monoclonal CA 15-3 antibody specifically binding to tumor marker CA 15-3 is immobilized; and a control line in which a control antibody is immobilized; and
d) an absorbent pad having one end overlapping the other end of the membrane to be capable of absorbing a liquid sample from the membrane.

2. The rapid kit of claim 1, wherein the sample pad, the gold conjugate pad, the membrane, and the absorbent pad are disposed in that order.

3. The rapid kit of claim 1, wherein the control antibody is an anti-mouse antibody (IgG).

4. The rapid kit of claim 1, wherein the biological sample is human whole blood, or plasma or serum isolated from whole blood.

5. A method for providing information for breast cancer diagnosis, the method comprising:
a) allowing a biological sample to be absorbed into the sample pad of the rapid kit for breast cancer diagnosis of claim 1;
b) allowing the biological sample to be developed to the gold conjugate pad, the membrane, and the absorbent pad from the sample pad absorbing the biological sample; and
c) identifying the signals from the test line and the control line of the membrane to determine the presence or absence of tumor marker CA 15-3.

6. The method of claim 5, wherein in step (c), the tumor marker CA 15-3 is determined to be present if color bands are shown in the test line and the control line of the membrane, and the tumor marker CA 15-3 is determined to be absent if a color band is shown in only the control line.
